(19) United States
(12) Patent Application Publication
(10) Pub. No.: US 2025/0282535 A1
(43) Pub. Date: Sep. 11, 2025

(54) PHARMACEUTICAL PACKAGES AND METHODS OF REDUCING FORMATION OF N-NITROSODIMETHYLAMINE

(71) Applicant: NOSTRUM PHARMACEUTICALS, LLC, Somerset, NJ (US)

(72) Inventors: Nirmal V. MULYE, Somerset, NJ (US); Anis Ahmed SHAIKH, Somerset, NJ (US)

(73) Assignee: NOSTRUM PHARMACEUTICALS, LLC, Somerset, NJ (US)

(21) Appl. No.: 18/546,089

(22) PCT Filed: Feb. 23, 2022
(86) PCT No.: PCT/US2022/017550

§ 371 Date: Aug. 10, 2023

Related U.S. Application Data
(60) Provisional application No. 63/153,310, filed on Feb. 24, 2021.

(51) Int. Cl. B65D 81/26; A61J 1/03; A61J 1/14; A61K 31/155; G01N 33/15

(52) U.S. Cl. CPC B65D 81/268; A61J 1/03; A61J 1/1468; A61K 31/155; G01N 33/15

(57) ABSTRACT

Systems and methods for reducing formation of NDMA are provided. A pharmaceutical package includes a pharmaceutical solid dosage packaging container. The pharmaceutical solid dosage packaging container includes one or more desiccants and pharmaceutical product comprising an active ingredient disposed in the container. The weight ratio of desiccant to active pharmaceutical ingredient is at least 1:150; and an NDMA level is no more than 30 ug NMDA per kg of pharmaceutical product for at least 3 months under accelerated storage conditions at about 40° C. and about 75% relative humidity. Method includes providing a desiccant to a container comprising a pharmaceutical product having an active pharmaceutical ingredient in a weight ratio of the desiccant to the API of at least 1:150 to maintain an NDMA level of no more than 30 ug NMDA per kg of the pharmaceutical product for at least 3 months under accelerated storage conditions.

### CROSS REFERENCE TO RELATED APPLICATIONS

The present application claims priority to and the benefit of U.S. App. No. 63/153,310, filed Feb. 24, 2021, which is hereby incorporated by reference in its entirety.

### FIELD OF DISCLOSURE

The present invention relates to pharmaceutical packages and methods of reducing formation of N-nitrosodimethylamine (NDMA) in a pharmaceutical product. More specifically, the invention relates to Metformin pharmaceutical packages and methods of reducing formation of NDMA in pharmaceutical product comprising Metformin active pharmaceutical ingredient.

### BACKGROUND

N-nitrosodimethylamine (NDMA) is a genotoxic and carcinogenic agent in animals and is classified as probably carcinogenic to humans (Class 2A carcinogen) by the World Health Organization's (WHO's) International Agency for Research on Cancer (IARC). NDMA may increase the risk of cancer if people are exposed to NDMA above an acceptable level and over a long period of time. Nitrosamine impurities which could be human carcinogens have been detected in drugs such as angiotensin II receptor blockers (ARBs), ranitidine, nizatidine, and metformin. Although some methods have been suggested for maintaining stability of a pharmaceutical product, existing methods of maintaining stability of a pharmaceutical product suffer from various disadvantages and might not also reduce NDMA formation for the drug chemistry of a specific pharmaceutical product.

Oxygen scavengers or desiccants have been used to try to maintain stability of a pharmaceutical product. But oxygen scavengers, such as iron based oxygen scavengers used for certain pharmaceutical products also add moisture controlled by salt slurries. Disadvantage or difficulty is balance between oxygen scavenger and humidity and balance between oxygen scavenger and amount of desiccant to remove humidity. Although iron oxygen scavengers might perform well for some or many pharmaceutical applications, oxygen scavengers cause the humidity in a bottle or container environment to be at 55 to 75% relative humidity, since oxygen consumption reaction requires humidity to operate. Although it is possible to dry a pharmaceutical bottle environment somewhat using a desiccant, the oxygen scavenger will, in general, also be dried by the desiccant and be less effective at removing the oxygen permeating through the bottle walls. The result is that the oxygen level in the bottle will not remain low enough to provide for stabilization of the pharmaceutically active ingredient over its entire shelf life.

Non-iron based oxygen scavengers that do not increase relative humidity have been marketed for use with pharmaceuticals. However, disadvantage of non-iron based oxygen scavengers might include limited absorption capacity (typically less than about 40-cc of oxygen), which is not adequate to provide for protection of pharmaceuticals in permeable packages for a typical shelf life of at least two years. Although it is possible, in theory, to use several such units to provide for adequate oxygen absorption on an ongoing basis, for common bottle sizes of 30-250 cc, the sheer number needed to maintain a low oxygen level during the shelf life of the pharmaceutical would generally preclude filling with dosage forms.

Furthermore, disadvantages or difficulty also exist regarding the type and amount of desiccant. If a desiccant does not have enough absorption capacity, a pharmaceutical product will not be adequately protected from moisture. If too much desiccant is included, a pharmaceutical product could become brittle and can crack. Thus, disadvantage or difficulty include selecting type and amount of desiccant which has enough absorption capacity to adequately protect pharmaceutical product from moisture but not too much desiccant to make a pharmaceutical product brittle or crack.

Further disadvantages or difficulties include finding optimal or suitable environment for a specific pharmaceutical product's chemistry for maintaining shelf life while also reducing NDMA formation. Thus, a need still exists for a pharmaceutical package and method of maintaining stability and also reducing NDMA in pharmaceutical products such as Metformin.

### BRIEF SUMMARY OF THE INVENTION

The present invention meets these and other needs by providing pharmaceutical package and method of reducing NDMA in pharmaceutical products such as Metformin. Applicant has unexpectedly discovered an unique pharmaceutical package and method with a type and amount of desiccant for Metformin's specific drug chemistry to reduce NDMA while also maintaining Metformin's stability and efficacy. Applicant has also unexpectedly discovered an unique pharmaceutical package and method with a type and amount of oxygen scavenger for Metformin's specific drug chemistry to reduce NDMA while also maintaining Metformin's stability and efficacy. Applicant has also unexpectedly discovered a balance between oxygen scavenger and humidity and balance between oxygen scavenger and amount of desiccant to remove humidity while maintaining stability for a respective specific pharmaceutical's chemistry while also reducing NDMA formation.

Accordingly, an aspect of the invention provides a pharmaceutical package comprising a pharmaceutical solid dosage packaging container. The pharmaceutical solid dosage packaging container includes one or more desiccants and pharmaceutical product comprising an active ingredient disposed in the container. The weight ratio of desiccant to active pharmaceutical ingredient is at least 1:150; and an NDMA level is no more than 30 ug NMDA per kg of pharmaceutical product for at least 3 months under accelerated storage or at least two years at room temperature. Accelerated conditions are at about 38° C.-42° C. and about 70-80% relative humidity.

A second aspect of the invention provides a pharmaceutical package comprising a pharmaceutical solid dosage packaging container. The pharmaceutical solid dosage packaging container includes one or more desiccants and a pharmaceutical product having Metformin active ingredient disposed in the container. The desiccant includes by weight about 55-65% Silica Gel (SiO₂) and about 35-45% activated Carbon; and the weight ratio of desiccant to Metformin active pharmaceutical ingredient is at least 1:150; and an NDMA level is no more than 30 ug NMDA per kg of pharmaceutical product for at least 3 months under accelerated storage or at least two years at room temperature.

A third aspect of the invention provides a pharmaceutical package comprising a pharmaceutical solid dosage packaging container. The pharmaceutical solid dosage packaging container includes one or more desiccants, one or more oxygen scavengers, and pharmaceutical product comprising an active ingredient disposed in the container. The weight ratio of desiccant to active pharmaceutical ingredient is at least 1:150; and weight ratio of oxygen scavenger to active pharmaceutical ingredient is at least 1:150. An NDMA level is no more than 30 ug NMDA per kg of pharmaceutical product for at least 3 months under accelerated storage conditions at about 38° C.-42° C. and about 70-80% relative humidity or at least two years at room temperature.

A fourth aspect of the invention provides a method of reducing formation of NDMA in a pharmaceutical product. The method includes providing a desiccant to a container comprising a pharmaceutical product having an active pharmaceutical ingredient in a weight ratio of the desiccant to the active pharmaceutical ingredient of at least 1:150 to maintain an NDMA level of no more than 30 ug NMDA per kg of the pharmaceutical product for at least 3 months under accelerated storage conditions at about 38° C.-42° C. and about 70-80% relative humidity or at least two years at room temperature.

A fifth aspect of the invention provides a method of reducing formation of NDMA in a pharmaceutical product. The method includes providing a desiccant to a container comprising pharmaceutical product comprising an active pharmaceutical ingredient in a weight ratio of the desiccant to the active pharmaceutical ingredient of at least 1:150; and providing an oxygen scavenger to the container in a weight ratio of the oxygen scavenger to the active pharmaceutical ingredient of at least 1:150 to maintain an NDMA level of no more than 30 ug NMDA per kg of pharmaceutical product for at least 3 months under accelerated storage conditions at about 38° C.-42° C. and about 70-80% relative humidity or at least two years at room temperature.

A sixth aspect of the invention provides a pharmaceutical package comprising a pharmaceutical solid dosage packaging container. The pharmaceutical solid dosage packaging container includes one or more oxygen scavengers and pharmaceutical product comprising an active ingredient disposed in the container. The weight ratio of oxygen scavenger to active pharmaceutical ingredient is at least 1:75. An NDMA level is no more than 30 ug NMDA per kg of pharmaceutical product for at least 3 months under accelerated storage conditions at about 38° C.-42° C. and about 70-80% relative humidity or at least two years at room temperature.

A seventh aspect of the invention provides a method of reducing formation of NDMA in a pharmaceutical product. The method includes providing an oxygen scavenger to a container comprising a pharmaceutical product having an active pharmaceutical ingredient in a weight ratio of the oxygen scavenger to the active pharmaceutical ingredient of at least 1:75 to maintain an NDMA level of no more than 30 ug NMDA per kg of the pharmaceutical product for at least 3 months under accelerated storage conditions at about 38° C.-42° C. and about 70-80% relative humidity or at least two years at room temperature.

These and other aspects, advantages, and salient features of the present invention will become apparent from the following detailed description, the accompanying drawings, and the appended claims.

### BRIEF DESCRIPTION OF THE FIGURE

FIG. 1 provides a flow chart illustrating a method of reducing formation of certain impurities in a pharmaceutical product as disclosed herein.

### DETAILED DESCRIPTION

In the following description, it is understood that terms such as “top,” “bottom,” “outward,” “inward,” and the like are words of convenience and are not to be construed as limiting terms.

Reference will now be made in detail to exemplary embodiments of the invention, which are illustrated in the accompanying figures and examples. Referring to the drawings in general, it will be understood that the illustrations are for the purpose of describing a particular embodiment of the invention and are not intended to limit the invention thereto.

Whenever a particular embodiment of the invention is said to comprise or consist of at least one element of a group and combinations thereof, it is understood that the embodiment may comprise or consist of any of the elements of the group, either individually or in combination with any of the other elements of that group. Furthermore, when any variable occurs more than one time in any constituent or in formula, its definition on each occurrence is independent of its definition at every other occurrence. Also, combinations of substituents and/or variables are permissible only if such combinations result in stable compounds.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. The exact values of all half integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.1% to 2.5%, etc. It will be understood that the sum of all weight % of individual components will not exceed 100%.

By “consist essentially” it is meant that the ingredients include only the listed components along with the normal impurities present in commercial materials and with any other additives present at levels which do not affect the operation of the embodiments disclosed herein, for instance at levels less than 5% by weight or less than 1% or even 0.5% by weight.

During an investigation for possible root causes of NDMA impurities in Metformin Hydrochloride Extended-Release Tablets, USP 750, Applicant unexpected discovered the effects of one or more desiccants and one or more oxygen scavengers on NDMA levels during stability studies. During the investigation, Applicant unexpected discovered that one or more desiccants and one or more oxygen scavengers reduces NDMA formation while retaining stability. Specifically, Applicant has unexpectedly discovered an unique pharmaceutical package and method with a type and amount of desiccant for Metformin's specific drug chemistry to reduce NDMA while also maintaining Metformin's stability and efficacy. Applicant has also unexpectedly discovered an unique pharmaceutical package and method with a type and amount of oxygen scavenger for Metformin's specific drug chemistry to reduce NDMA while also maintaining Metformin's stability and efficacy. Applicant has also unexpectedly discovered a balance between oxygen scavenger and humidity and balance between oxygen scavenger and amount of desiccant to remove humidity while maintaining stability for a respective specific pharmaceutical's chemistry while also reducing NDMA formation.

An embodiment of the invention provides a pharmaceutical package comprising a pharmaceutical solid dosage packaging container. The pharmaceutical solid dosage packaging container includes one or more desiccants disposed in the container; and one or more pharmaceutical products comprising an active ingredient disposed in the container disposed in the container. The weight ratio of desiccant to active pharmaceutical ingredient is at least 1:150; and NDMA level is no more than 30 ug NMDA per kg of pharmaceutical product for at least 3 months under accelerated storage conditions or at least two years at room temperature. In an embodiment, the accelerated storage conditions include temperature in a range of about 38-42° C. or about 40-42° C. In another embodiment, the accelerated storage conditions include relative humidity in a range of about 70% to about 80% relative humidity. In another embodiment, the accelerated storage conditions include temperature at about 38° C. to about 42° C. and about 70-80% relative humidity. It should be appreciated and understood that embodiments of about 70% to about 80% relative humidity specifically disclose a percentage of 70.1%, 71%, 71.5%, 72.0%, 72.5%, and 73%, etc. Additionally, a range of about 70% to about 80% relative humidity includes subsets of the original range such as but not limited to from 70.5% to 75%, from 71% to 75%, from 7.5% to 80%, etc. It should also be appreciated and understood that embodiments of temperature at about 38° C. to about 42° C. specifically disclose a temperature of about 38.1° C., 38.5° C., 39° C., 39.5° C., 40° C., 40.5° C., 41° C., 41.5° C., etc. Additionally, a range of about 38° C. to about 42° C. temperature includes subsets of the original range such as but not limited to from 38.1° C. to 38.9° C., from 39° C. to 40° C., from 41° C. to 42° C., etc.

In an embodiment, NDMA level is no more than 30 ug NMDA per kg of pharmaceutical product at least two years at room temperature. In another embodiment, NDMA level is no more than 30 ug NMDA per kg of pharmaceutical product for at least 3 months under accelerated storage conditions at about 38° C.-42° C. and about 70-80% relative humidity. In a particular embodiment, NDMA level is no more than 30 ug NMDA per kg of pharmaceutical product for at least 3 months under accelerated storage conditions at about 40-42° C. and about 70-75% relative humidity. In another embodiment, NDMA level is no more than 30 ug NMDA per kg of pharmaceutical product for at least 3 months under accelerated storage conditions at 40° C. and 75% relative humidity.

### Active Pharmaceutical Ingredient (API)

In an embodiment, the active pharmaceutical ingredient includes angiotensin II receptor blockers (ARBs), ranitidine, nizatidine, and Metformin, either individually or in a combination of two or more thereof. In another embodiment, the active pharmaceutical ingredient includes one or more secondary amine group. In a particular embodiment, the active pharmaceutical ingredient includes biguanides such as but not limited to Metformin. In a particular embodiment, the active pharmaceutical ingredient includes metformin. In another embodiment, the active pharmaceutical ingredient includes H2 blockers and angiotensin II receptor blockers. In another embodiment, the active pharmaceutical ingredient includes ranitidine. In yet another embodiment, the active pharmaceutical ingredient includes nizatidine.

### Dessicant

In an embodiment, the desiccant includes such as but not limited to silica gel, clay, molecular sieves, either individually or in a combination of two or more thereof. In a particular embodiment, the desiccant includes silica gel. In yet another embodiment, the desiccant includes by weight about 55-65% Silica Gel (SiO₂) and about 35-45% activated Carbon. In another embodiment, the desiccant includes by weight about 55-65% Silica Gel (SiO₂) and about 35-45% activated Carbon. In a particular embodiment, the desiccant substantially comprises by weight about 55-65% Silica Gel (SiO₂) and about 35-45% activated Carbon.

In an embodiment, the weight ratio of desiccant to active pharmaceutical ingredient is at least 1:150. The weight ratio of desiccant to active pharmaceutical ingredient may vary within the scope of the invention. In another embodiment, the weight ratio of desiccant to active pharmaceutical ingredient is at least 1:75. In another embodiment, the active pharmaceutical ingredient includes Metformin; and the desiccant includes by weight about 55-65% Silica Gel (SiO₂) and about 35-45% activated Carbon; and the weight ratio of desiccant to metformin is at least 1:150 In another embodiment, the active pharmaceutical ingredient includes Metformin; and the desiccant includes by weight about 55-65% Silica Gel (SiO₂) and about 35-45% activated Carbon; and the weight ratio of desiccant to metformin is at least 1:75. In yet another embodiment, the active pharmaceutical ingredient includes Metformin; and the desiccant has by weight about 55-65% Silica Gel (SiO₂) and about 35-45% activated Carbon; and the weight ratio of desiccant to metformin is at least 1:150. In yet another embodiment, the active pharmaceutical ingredient includes Metformin; and the desiccant has by weight about 55-65% Silica Gel (SiO₂) and about 35-45% activated Carbon; and the weight ratio of desiccant to metformin is at least 1:75.

In an embodiment, the pharmaceutical solid dosage packaging container has a first slot for desiccant.

In an embodiment, the pharmaceutical solid dosage packaging container includes a plurality of desiccants. When more than one desiccant is present, the plurality of desiccants may have various characteristics. The plurality of desiccants may differ from each other or be the same. For example, the desiccants may include silica gel, clay, molecular sieves, either individually or in a combination of two or more. In one embodiment, the desiccants may be chemically or physically separate and distinct from each other. In another embodiment, the desiccants may be chemically or physically reacted.

It should be appreciated that the desiccant includes the reaction product of the one or more desiccants with each other and the reaction product of the one or more desiccants with the one or more active pharmaceutical ingredients.

### Oxygen Scavengers

In an embodiment, the pharmaceutical solid dosage packaging container further includes one or more oxygen scavengers disposed in the container. Examples of oxygen scavengers includes such as but not limited to Pharmakeep™. In another non-limiting embodiment for illustration and not limitation, oxygen scavenger may be enclosed in a porous sachet that can be placed within the pharmaceutical solid dosage packaging container. Examples of oxygen scavengers include such as alkaline solution of pyrogallic acid in an air-tight vessel, mixture of iron powder and sodium chloride, either individually or in a combination of two or more. In another embodiment, oxygen-scavenging compounds can be incorporated directly into a packaging material itself. These materials include flexible films, rigid plastics, and liners in closures. In another embodiment, oxygen scavenger includes (I) metal alloy comprising (A) one or more transition metals such as manganese, iron, platinum, and copper group metals and (B) one or more metals such as aluminum, zinc, tin, lead, magnesium, and silicon and treated with an acidic or alkaline aqueous solution to elute and remove at least a part of the component (B); and (II) a thermoplastic resin. In an embodiment, the weight ratio of oxygen scavenger to active pharmaceutical ingredient is at least 1:150. In an embodiment, the pharmaceutical solid dosage packaging container includes a plurality of oxygen scavengers disposed in the container. The plurality of oxygen scavengers may have various characteristics. The plurality of oxygen scavengers may differ from each other or be the same. In one embodiment, the oxygen scavengers may be chemically or physically separate and distinct from each other. In another embodiment, the oxygen scavengers may be chemically or physically reacted.

It should be appreciated that the pharmaceutical solid dosage packaging container also includes the reaction product of the one or more oxygen scavengers with each other, the reaction product of the one or more desiccants with the one or more oxidation scavengers, as well as the reaction product of the one or more oxygen scavengers and or one or more desiccants with the active pharmaceutical ingredient.

In an embodiment, the pharmaceutical solid dosage packaging container has a slot for desiccant. In another embodiment, the pharmaceutical solid dosage packaging container has a slot for oxygen scavengers. In another embodiment, the pharmaceutical solid dosage packaging container has a slot for desiccant and a slot for oxygen scavengers.

Another embodiment provides a pharmaceutical package comprising a pharmaceutical solid dosage packaging container. The pharmaceutical solid dosage packaging container includes one or more oxygen scavengers and pharmaceutical product comprising an active ingredient disposed in the container. The weight ratio of oxygen scavenger to active pharmaceutical ingredient is at least 1:150; and an NDMA level is no more than 30 ug NMDA per kg of pharmaceutical product for at least 3 months under accelerated storage conditions or at least two years at room temperature. In an embodiment, the accelerated storage conditions include temperature in a range of about 38-42° C. or about 40-42° C. In another embodiment, the accelerated storage conditions include relative humidity in a range of about 70-80% relative humidity. In another embodiment, the accelerated storage conditions include temperature at about 38° C.-42° C. and about 70-80% relative humidity.

In an embodiment, the pharmaceutical solid dosage packaging container further includes one or more desiccants in addition to the oxygen scavenger. In an embodiment, the pharmaceutical solid dosage packaging container has a slot for desiccant. In another embodiment, the pharmaceutical solid dosage packaging container has a slot for oxygen scavengers. In another embodiment, the pharmaceutical solid dosage packaging container has a slot for desiccant and a slot for oxygen scavengers.

Another embodiment provides a pharmaceutical package comprising a pharmaceutical solid dosage packaging container. The pharmaceutical solid dosage packaging container includes activated charcoal and pharmaceutical product comprising an active ingredient disposed in the container. The weight ratio of the activated charcoal to the active pharmaceutical ingredient is greater than or equal to 1:225; and an NDMA level is no more than 30 ug NMDA per kg of pharmaceutical product for at least 3 months under accelerated storage conditions or at least two years at room temperature. In an embodiment, the accelerated storage conditions include temperature in a range of about 38-42° C. or about 40-42° C. In another embodiment, the accelerated storage conditions include relative humidity in a range of about 70-80% relative humidity. In another embodiment, the accelerated storage conditions include temperature at about 38° C.-42° C. and about 70-80% relative humidity.

In an embodiment, the pharmaceutical solid dosage packaging container further includes one or more desiccants in addition to the activated charcoal. In another embodiment, the pharmaceutical solid dosage packaging container further includes silica gel desiccant disposed in the container in addition to the activated charcoal. In yet another embodiment, the pharmaceutical solid dosage packaging container further includes silica gel desiccant and oxygen scavenger disposed in the container in addition to the activated charcoal.

Although the examples are described with NDMA, embodiments of the invention include reducing impurities such as but not limited to Nitrosodimethylamine (NDMA), N-Nitrosodiethylamine (NDEA), N-Nitrosomethylphenylamine (NMPA), N-Nitrosodiisopropylamine (NDIPA), N-Nitrosoisopropylethylamine (NIPEA), and N-Nitroso-N-methyl-4-aminobutyric Acid (NMBA) etc. either individually or in combinations of two or more thereof. Embodiments of the invention include maintaining the impurity level below a designated level for impurities such as but not limited to Nitrosodimethylamine (NDMA), N-Nitrosodiethylamine (NDEA), N-Nitrosomethylphenylamine (NMPA), N-Nitrosodiisopropylamine (NDIPA), N-Nitrosoisopropylethylamine (NIPEA), and N-Nitroso-N-methyl-4-aminobutyric Acid (NMBA) etc. either individually or in combinations of two or more thereof.

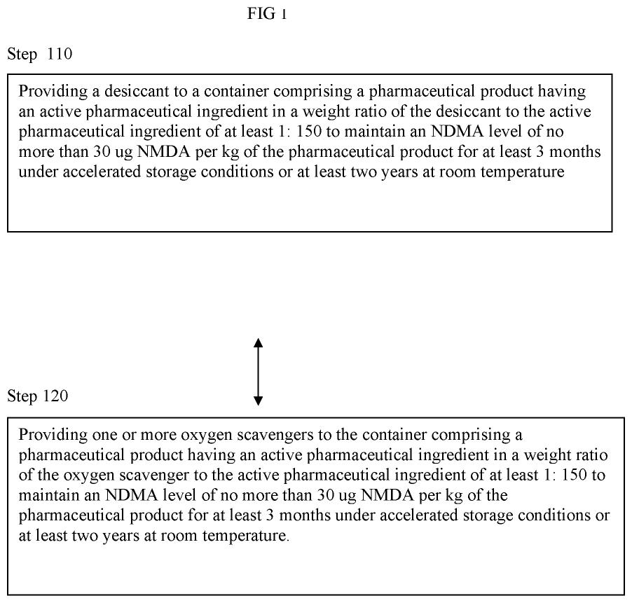

Another embodiment provides a method of reducing formation of NDMA in a pharmaceutical product as depicted in FIG. 1. The method includes Step 110 (FIG. 1) providing one or more desiccants to a container comprising a pharmaceutical product having an active pharmaceutical ingredient in a weight ratio of the desiccant to the active pharmaceutical ingredient of at least 1:150 to maintain an NDMA level of no more than 30 ug NMDA per kg of the pharmaceutical product for at least 3 months under accelerated storage conditions or at least two years at room temperature.

As discussed above, in an embodiment, the accelerated storage conditions include temperature in a range of about 38-42° C. or about 40-42° C. In another embodiment, the accelerated storage conditions include relative humidity in a range of about 70% to about 80% relative humidity. In another embodiment, the accelerated storage conditions include temperature at about 38° C. to about 42° C. and about 70-80% relative humidity. It should be appreciated and understood that embodiments of about 70% to about 80% relative humidity specifically disclose a percentage of 70.1%, 71%, 71.5%, 72.0%, 72.5%, and 73%, etc. Additionally, a range of about 70% to about 80% relative humidity includes subsets of the original range such as but not limited to from 70.5% to 75%, from 71% to 75%, from 7.5% to 80%, etc. It should also be appreciated and understood that embodiments of temperature at about 38° C. to about 42° C. specifically disclose a temperature of about 38.1° C., 38.5° C., 39° C., 39.5° C., 40° C., 40.5° C., 41° C., 41.5° C., etc. Additionally, a range of about 38° C. to about 42° C. temperature includes subsets of the original range such as but not limited to from 38.1° C. to 38.9° C., from 39° C. to 40° C., from 41° C. to 42 C, etc.

The method is not limited by a sequence of when and how the desiccant is provided. The desiccant may be provided to the solid dosage packaging container at one time in one unit or increment or repeatedly in multiple units or amount. For example, in an embodiment, the desiccant is provided in a weight ratio of desiccant to active pharmaceutical ingredient of at least 1:150; and the total amount of desiccants may be provided in one unit at one time or increment or repeatedly in multiple units. In another embodiment, a plurality of desiccants may be provided to the solid dosage packaging container. The plurality of desiccants may differ from each other or be the same. The total sum amount of the plurality of desiccants may be provided in one unit at one time or increment or repeatedly in multiple units.

In an embodiment, the method further also includes Step 120 (FIG. 1) providing one or more oxygen scavenger to the solid dosage packaging container comprising the pharmaceutical product having an active pharmaceutical ingredient in a weight ratio of the oxygen scavenger to the active pharmaceutical ingredient of at least 1:150 to maintain an NDMA level of no more than 30 ug NMDA per kg of the pharmaceutical product for at least 3 months under accelerated storage conditions or at least two years at room temperature.

The method is not limited by a sequence of when and how the oxygen scavenger is provided. The oxygen scavenger may be provided to the solid dosage packaging container at one time in one unit or increment or repeatedly in multiple units or amount. For example, in an embodiment, the oxygen scavenger is provided in a weight ratio of oxygen scavenger to active pharmaceutical ingredient of at least 1:150; and the total amount of oxygen scavenger may be provided in one unit at one time or increment or repeatedly in multiple units. In another embodiment, a plurality of oxygen scavengers may be provided to the solid dosage packaging container. The plurality of oxygen scavengers may be the same or differ from each other. The total sum amount of the plurality of oxygen scavenger may be provided in one unit at one time or increment or repeatedly in multiple units.

The method is also not limited by a sequence, frequency, and how the desiccant and oxygen scavenger are provided in relation to each other. The oxygen scavenger and desiccant can be either sequentially or simultaneously provided to the solid dosage packaging container. The oxygen scavenger may be provided before, during, or after the desiccant is provided.

The method is also not limited by the form of how the desiccant and oxygen scavenger are provided to the solid dosage packaging container. Examples of form include, but are not limited to, particles, grains, pellets, powders, extrudate, spheres, granules, either individually or in a combination of two or more.

In another embodiment, the method includes providing one or more oxygen scavenger to the solid dosage packaging container comprising a pharmaceutical product having an active pharmaceutical ingredient in a weight ratio of the oxygen scavenger to the active pharmaceutical ingredient of at least 1:150 to maintain an NDMA level of no more than 30 ug NMDA per kg of the pharmaceutical product for at least 3 months under accelerated storage conditions or at least two years at room temperature.

As discussed above, in an embodiment, the accelerated storage conditions include temperature in a range of about 38-42° C. or about 40-42° C. In another embodiment, the accelerated storage conditions include relative humidity in a range of about 70% to about 80% relative humidity. In another embodiment, the accelerated storage conditions include temperature at about 38° C. to about 42° C. and about 70-80% relative humidity. It should be appreciated and understood that embodiments of about 70% to about 80% relative humidity specifically disclose a percentage of 70.1%, 71%, 71.5%, 72.0%, 72.5%, and 73%, etc. Additionally, a range of about 70% to about 80% relative humidity includes subsets of the original range such as but not limited to from 70.5% to 75%, from 71% to 75%, from 7.5% to 80%, etc. It should also be appreciated and understood that embodiments of temperature at about 38° C. to about 42° C. specifically disclose a temperature of about 38.1° C., 38.5° C., 39° C., 39.5° C., 40° C., 40.5° C., 41° C., 41.5° C., etc. Additionally, a range of about 38° C. to about 42° C. temperature includes subsets of the original range such as but not limited to from 38.1° C. to 38.9° C., from 39° C. to 40° C., from 41° C. to 42 C, etc.

The method is not limited by a sequence of when and how the oxygen scavenger is provided. The oxygen scavenger may be provided to the solid dosage packaging container at one time in one unit or increment or repeatedly in multiple units or amount. For example, in an embodiment, the oxygen scavenger is provided in a weight ratio of oxygen scavenger to active pharmaceutical ingredient of at least 1:150; and the total amount of oxygen scavenger may be provided in one unit at one time or increment or repeatedly in multiple units. In another embodiment, a plurality of oxygen scavengers may be provided to the solid dosage packaging container. The plurality of oxygen scavengers may be the same or differ from each other. The total sum amount of the plurality of oxygen scavenger may be provided in one unit at one time or increment or repeatedly in multiple units.

In a particular embodiment, the method further includes also providing one or more desiccants to the container comprising a pharmaceutical product having an active pharmaceutical ingredient in a weight ratio of the desiccant to the active pharmaceutical ingredient of at least 1:150 to maintain an NDMA level of no more than 30 ug NMDA per kg of the pharmaceutical product for at least 3 months under accelerated storage conditions or at least two years at room temperature.

The method is not limited by a sequence of when and how the desiccant is provided. The desiccant may be provided to the solid dosage packaging container at one time in one unit or increment or repeatedly in multiple units or amount. For example, in an embodiment, the desiccant is provided in a weight ratio of desiccant to active pharmaceutical ingredient of at least 1:150; and the total amount of desiccants may be provided in one unit at one time or increment or repeatedly in multiple units. In another embodiment, a plurality of desiccants may be provided to the solid dosage packaging container. The plurality of desiccants may differ from each other or be the same. The total sum amount of the plurality of desiccants may be provided in one unit at one time or increment or repeatedly in multiple units.

The method is also not limited by a sequence, frequency, and how the desiccant and oxygen scavenger are provided in relation to each other. The oxygen scavenger and desiccant can be either sequentially or simultaneously provided to the solid dosage packaging container. The oxygen scavenger may be provided before, during, or after the desiccant is provided.

The method is also not limited by the form of how the desiccant and oxygen scavenger are provided to the solid dosage packaging container. Examples of form include, but are not limited to, particles, grains, pellets, powders, extrudate, spheres, granules, either individually or in a combination of two or more.

In an embodiment, the method further detecting the amount of NDMA in the container comprising the active pharmaceutical ingredient and desiccant at time zero; and detecting the amount of NDMA in the container comprising the active pharmaceutical ingredient and desiccant after 30 days;

detecting the amount of NDMA in a container comprising the active pharmaceutical ingredient without desiccant at time zero; and detecting the amount of NDMA in the container comprising the active pharmaceutical ingredient without desiccant after 30 days; and comparing the amount of NDMA in the container comprising the active pharmaceutical ingredient and desiccant with the amount of NDMA in the container comprising the active pharmaceutical ingredient without desiccant.

In another embodiment, the method further includes:
detecting the amount of NDMA in the container comprising the active pharmaceutical ingredient and desiccant after 60 days;
detecting the amount of NDMA in the container comprising the active pharmaceutical ingredient without desiccant after 60 days; and
comparing the amount of NDMA in the container comprising the active pharmaceutical ingredient and desiccant with the amount of NDMA in the container comprising the active pharmaceutical ingredient without desiccant.

In another embodiment, the method further includes:
detecting the amount of NDMA in the container comprising the active pharmaceutical ingredient and desiccant after 90 days;
detecting the amount of NDMA in the container comprising the active pharmaceutical ingredient without desiccant after 90 days; and
comparing the amount of NDMA in the container comprising the active pharmaceutical ingredient and desiccant with the amount of NDMA in the container comprising the active pharmaceutical ingredient without desiccant.

In an embodiment, N-nitrosodimethylamine (NDMA) was measured by the following non-limiting technique as known in the art for measuring N-nitrosodimethylamine impurities in Metformin raw material and Drugs containing Metformin.

### Procedure:

1. Balance 250 mg sample and note down the accurate weight.
2. Spike the sample with spiking standard (100 μg/L NDMA Standard in water).
3. Add exactly 5 ml 1N HCl and shake or sonicate till sample is completely dissolved.
4. Add exactly 5 ml of dichloromethane.
5. Shake for 10 min.
6. Centrifuge for 10 min at 4000 rpm to separate the phases.
7. Transfer dichloromethane layer (bottom) to a GC vial.
8. Measure by Gas Chromatography (GS)-Mass Spectroscopy and calibrate the system with 0.5, 1, 5, 10, 20 ppb standards of NDMA and NDMA

### Equipment:

1. GC-MS/MS system
2. Two connected HP-5 ms Ultra Inert columns (Agilent Part No. 19091S-431UI) −15 m×250 μm×0.25 μm.

### Equipment Conditions:

GC-Parameters:
Injection Volume: 1.8 μL
Injector Temp: 250° C.
Flow first column: 0.994 ml/min
Flow second column: 1.195 ml/min
Quench Gas: 4 ml/min (He)
Collision Gas: 1.5 ml/min (N2)
Oven Temp Program: 50° C. (4.5 min), 120° C./min to 250° C., 1 min (Post run at 280° C.)

MS/MS-Parameters:
Source Temp: 280° C.
Gain Factor: 10

### EXAMPLES

The following examples illustrate the features of embodiments of the invention and are not intended to limit the invention thereto. Various compositions were prepared and tested for their ability to reduce NDMA formation. Non-limiting samples along with their compositions is shown below (such as Table 3).

It should be understood that it is within and included in the scope of the invention to adjust the type and amount of desiccant and the type and amount of oxygen scavenger.

FDA's recommended limit of 96 ng/day for N-nitrosodimethylamine (NDMA) Impurity. FDA's recommended limit of 96 ng/day for NDMA translated to no more than (NMT) of 30 ug NDMA/kg of final product (pharmaceutical tablet product)

Metformin Hydrochloride Extended Release Tablet 500 mg and 750 mg product characteristics are presented in Table 1. Dose range may be initiated at 500 mg API once daily up to maximum daily dose (MDD) of 2000 mg API/day.
2000 mg API/day can be taken based on 4 tablets with 500 mg API per tablet (unit) based on:
A tablet (unit) having 500 mg API x 4 tablets with 500 mg API/tablet=2000 mg API Each tablet having 500 mg API weights 800 mg.
Thus, 4 tablets x 800 mg/weight of whole tablet=3200 mg=3.2 g
Thus, 3.2 g of finished product (4 tablets) is based on 4 units (4 tablets of final product Metformin Hydrochloride Extended Release Tablet 500 mg)

### Based On:

No more than 96 ng ND MA/day÷3.2 g finished product tablet/day
=No more than 96 ng NDMA/day×day/3.2 g finished product
=no more than 30 ng NDMA/g finished product
=no more than 30 ug NDMA/kg finished product

TABLE 1
Product characteristics based on the recommendation
for the products covered by this risk assessment.

| Parameter | Product Characteristics | |
|---|---|---|
| Dosage form | Un-coated tablets (oral route) | |
| Strength | 500 mg and 750 mg Metformin | |
| Unit (1 tablet) | 800 mg for 500 mg strength | |
| Weight | 1200 mg for 750 mg strength | |
| Max daily dose | 2000 | mg/day |
| API (mg/day) | | |
| Max daily | 3.2 | g |

| | |
|---|---|
| product weight (g) | |
| Clinical indication | Metformin Hydrochloride Extended Release |
| | Tablet 500 mg and 50 mg is indicated as an |
| | adjunct to diet and exercise to improve glycemic |
| | control in adults with type 2 diabetes mellitus. |
| Production Facility | Multipurpose production facility |
| Equipment material | SS316 |
| Drug Substance | USP compliant Drug Substnace |
| Water | Purified water (USP/NF grade) |

TABLE 2
Qualitative-quantitative composition of Metformin Hydrochloride
Extended Release Tablets USP, 500 mg and 750 mg

| | | |
|---|---|---|
| Component | Total weight (mg) | g/MDD |

| | | | |
|---|---|---|---|
| Metformin Hydrochloride USP | 500 | 750 | 2.000 |
| Total weight of 1 unit i.e. | 800 | 1200 | 3.200 |
| 1 tablet with other ingredients | | | |

The following 4 respective studies shown in Table 3 were conducted to respectively assess effects of desiccant and oxygen scavenger on NDMA formation on 100 Metformin Hydrochloride Extended-Release Tablets USP 750 mg (100 metformin tablets having 75 g Metformin API by weight and total weight of 120 g).

Commercial batch of Metformin Hydrochloride Extended-Release Tablets, USP 750 mg was used for this study. Stability testing was carried out to provide evidence of how the quality of the manufactured tablets (especially NDMA Impurity) may change with time under the influence of environmental factors such as temperature and humidity coupled with presence and absence of Desiccant and/or Oxygen scavengers.

Desiccant by Clariant™ was used. 1 unit of Desiccant weighs 1 g and has by weight 60% silica gel and 40% activated carbon. The unit of Desiccant is provided in a can. Oxygen scavenger such as by Pharmakeep® (PharmaKeep® can protect against oxidative degradation and extend the shelf life of pharmaceuticals). Oxygen and moisture in the packaging could increase NDMA Impurity; PharmaKeep® can absorb oxygen and moisture. Providing Oxygen scavenger such as by Pharmakeep® in the packaging can help maintain low oxygen and low humidity and control the formation of N-nitrosodimethylamine (NDMA) impurity during shelf life. Pharmakeep® absorbs oxygen independently of moisture and combinations help control moisture, gas, and odors simultaneously. Can 17.9×22.5 CD-2.15G Pharmakeep® has capacity to absorb Oxygen (O₂) of more than 20 mL at 25° C. (+/−2° C.), 500 mL air, 7 days and moisture absorption capacity of more than 0.15 g water at 25° C. (+/−2° C.), 75% RH (+/−5%), 7 days.

One (1) commercial batch of Metformin Hydrochloride Extended-Release Tablets USP, 750 mg (Batch no. MET201501) was manufactured per approved master production record and the stability study was conducted using following packaging configurations.

Trial 1—Stability study of packaged product Metformin Hydrochloride Extended-release Tablets, USP 750 mg 100 count with one (1) Oxygen Scavenger (1 unit of 2.15 g Pharmakeep® canister made up of polypropylene containing Pharmakeep® granules) and one (1) Desiccant (1 unit of 1 g Desiccant having by weight 60% silica gel and 40% activated carbon) in a 200 cc high density polyethylene plastic (HDPE) bottle heat sealed with 45 mm Polypropylene closure.

Trial 2—Stability study of packaged product Metformin Hydrochloride Extended-release Tablets, USP 750 mg 100 count with two (2) Oxygen Scavenger (1 unit weighs 2.15 g Pharmakeep®) in a 200 cc HDPE container heat sealed with 45 mm Polypropylene closure.

Trial 3—Stability study of packaged product Metformin Hydrochloride Extended-release Tablets, USP 750 mg 100 count with two (2) Desiccants (1 unit weighs 1 g Desiccant having by weight 60% silica gel and 40% activated carbon) in a 200 cc HDPE container heat sealed with 45 mm Polypropylene closure.

Trial 4 control—Stability study of packaged product Metformin Hydrochloride Extended-release Tablets, USP 750 mg, 100 count without Oxygen Scavenger and without Desiccant in a 200 cc HDPE container heat sealed with 45 mm Polypropylene closure.

In stability studies, Metformin Hydrochloride Extended-Release Tablets, USP 750 were evaluated by testing NDMA Impurity. At the beginning (0 Month) the N-nitrosodimethylamine (NDMA) Impurity levels were found to be very low and well within finished product and stability specifications established for this product. NDMA Impurity limit for Metformin Hydrochloride Extended-Release Tablets is NMT 30 μg/kg and are in line with

At the end of 2 months, the NDMA Impurity of Metformin Hydrochloride Extended-Release tablets, USP in a high density polyethylene plastic container (bottle) without any desiccant or oxygen scavenger increased beyond finished product specifications and stability study specification of NMT 30 μg/kg due to the presence of moisture and oxygen into the containers.

However, at the end of 2 months, not much of an increase in N-nitrosodimethylamine (NDMA) impurity was found in the Metformin Hydrochloride Extended-Release tablets 750 mg after storage in high density polyethylene plastic container stored with addition of desiccant alone as well as with addition of oxygen scavenger alone and with combination of both (Table 3). Also, at the end of 3 months, not much of an increase in N-nitrosodimethylamine (NDMA) impurity was found in the Metformin Hydrochloride Extended-Release tablets 750 mg after storage in high density polyethylene plastic container stored with addition of desiccant alone as well as with addition of oxygen scavenger alone and with combination of both (Table 3).
Controlled Room temperature (CRT) conditions: 25±2° C. temperature/60±5% relative humidity
Intermediate Conditions: 30±2° C. temperature/65±5% relative humidity and
Accelerated Conditions: 40±2° C. temperature/75±5% relative humidity

TABLE 3
N-Nitrosodimethylamine (NDMA) Impurity Results at 0, 1, 2, and 3 Months

| | | | | |
|---|---|---|---|---|
| | | | | Trial 4 |
| | | | | Control |
| | Trial 1 | | | No Oxygen |
| | With Oxygen | Trial 2 | Trial 3 | Scavenger |
| Time Point | Scavenger and | With Oxygen | With | and |
| Condition | Desiccant | Scavenger | Desiccant | No Desiccant |

Specification - NMT 30 μg/kg (LOQ 10 μg/kg)

| | | | | |
|---|---|---|---|---|
| 0 Month | Below LOQ | Below LOQ | Below LOQ | Below LOQ |
| | (3.9 μg/kg) | (4.2 μg/kg) | (3.0 μg/kg) | (4.3 μg/kg) |
| 1 Month (25 ± 2° C. | Below LOQ | Below LOQ | Below LOQ | Below LOQ |
| temperature/60 ± 5% | (4.9 μg/kg) | (6.3 μg/kg) | (3.0 μg/kg) | (5.9 μg/kg) |
| relative humidity) | | | | |
| 1 Month (40 ± 2° C. | Below LOQ | 13.4 μg/kg | Below LOQ | 20.7 μg/kg |
| temperature/75 ± 5% | (9.4 μg/kg) | | (9.4 μg/kg) | |
| relative humidity) | | | | |
| 1 Month (30 ± 2° C. | Below LOQ | Below LOQ | Below LOQ | Below LOQ |
| temperature/65 ± 5% | (4.1μg/kg) | (6.6 μg/kg) | (4.2 μg/kg) | (8.5 μg/kg) |
| relative humidity) | | | | |
| 2 Months (25 ± 2° C. | Below LOQ | Below LOQ | Below LOQ | Below LOQ |
| temperature/60 ± 5% | (4.6 μg/kg) | (6.9 μg/kg) | (4.1 μg/kg) | (6.1 μg/kg) |
| relative humidity) | | | | |
| 2 Months (40 ± 2° C. | 13.2 μg/kg | 20.6 μg/kg | 11.3 μg/kg | 32.1 μg/kg |
| temperature/75 ± 5% | | | | |
| relative humidity) | | | | |
| 2 Months (30 ± 2° C. | Below LOQ | Below LOQ | Below LOQ | 13.0 μg/kg |
| temperature/65 ± 5% | (5.7 μg/kg) | (9.1 μg/kg) | (5.7 μg/kg) | |
| relative humidity) | | | | |
| 3 Months (25 ± 2° C. | Below LOQ | Below LOQ | Below LOQ | Below LOQ |
| temperature/60 ± 5% | (10 μg/kg) | (10 μg/kg) | (10 μg/kg) | (10 μg/kg) |
| relative humidity) | | | | |
| 3 Months (40 ± 2° C. | 10 μg/kg | 21.0 μg/kg | 10 μg/kg | 40.2 μg/kg |
| temperature/75 ± 5% | | | | |
| relative humidity) | | | | |
| 3 Months (30 ± 2° C. | Below LOQ | Below LOQ | Below LOQ | 15.7 μg/kg |
| temperature/65 ± 5% | (10 μg/kg) | (11.2 μg/kg) | (10 μg/kg) | |
| relative humidity) | | | | |

LOQ means Limit of Quantitation.

Based on the NDMA Impurity results obtained at the end of 2 months stability studies, following conclusions are drawn.

At the end of 2 months, Trial 1 data (in Table 3) with addition of both desiccant (1 unit of 1 g Desiccant having by weight 60% silica gel and 40% activated carbon) and Oxygen Scavenger (1 unit of 2.15 g Pharmakeep®) into the packaged product of Metformin Hydrochloride Extended-Release tablets significantly reduces the formation of NDMA. Trial 1 Stability study was carried out providing one (1) Oxygen Scavenger (1 Pharmakeep® unit weighing 2.15 G) and also one (1) desiccant unit (1 desiccant unit weighing 1 g) to packaged product Metformin Hydrochloride Extended-release Tablets, USP 750 mg 100 count in a 200 cc HDPE bottle heat sealed with 45 mm Polypropylene closure.

At the end of 2 months, Trial 2 data (in Table 3) with addition of Oxygen Scavenger (Pharmakeep®) alone into the packaged product of Metformin Hydrochloride Extended-Release tablets reduces the formation of NDMA. Trial 2 Stability study was carried out providing two (2) Oxygen Scavenger (Pharmakeep® each unit weighing 2.15 g with 2 units weighing total of 4.30 g) to packaged product Metformin Hydrochloride Extended-release Tablets, USP 750 mg 100 count in a 200 cc HDPE container heat sealed with 45 mm Polypropylene closure.

At the end of 2 months, Trial 3 data (in Table 3) with addition of desiccant (60SG/40CB) alone without oxygen scavenger into packaged product of Metformin Hydrochloride Extended-Release tablets significantly reduces the formation of NDMA. Trial 3 Stability study was carried out providing 2 g desiccant (2 units i.e. cans weighing 1 g each with total 2 g Desiccant having by weight 60% silica gel and 40% activated carbon) to packaged product Metformin Hydrochloride Extended-release Tablets, USP 750 mg 100 count in a 200 cc HDPE bottle heat sealed with 45 mm Polypropylene closure.

At the end of 2 months, Trial 4 control data (in Table 3) demonstrates that presence of moisture and air/oxygen into the packaged product facilitates the formation of NDMA Impurity in Metformin Hydrochloride Extended-Release tablets. Trial 4 control stability study was carried out without any desiccant and without any oxygen scavenger on packaged product Metformin Hydrochloride Extended-release Tablets, USP 750 mg, 100 count in a 200 cc HDPE container heat sealed with 45 mm Polypropylene closure.

At the end of 2 months of stability studies, the NDMA Impurity levels of packaged product with either the desiccant and/or oxygen scavenger were found to be controlled within the stability study specifications (NMT 30 μg/kg).

Based on the NDMA Impurity results obtained at the end of 3 months stability studies, following conclusions are drawn.

At end of 3 months, Trial 1 data (in Table 3) with addition of both desiccant (1 unit of 1 g Desiccant having by weight 60% silica gel and 40% activated carbon) and Oxygen Scavenger (1 unit of 2.15 g Pharmakeep®) into the packaged product of Metformin Hydrochloride Extended-Release tablets significantly reduces the formation of NDMA. Trial 1 Stability study was carried out providing one (1) Oxygen Scavenger unit (1 Pharmakeep® unit weighing 2.15G) and also one (1) desiccant unit (1 desiccant unit weighing 1 g) to packaged product Metformin Hydrochloride Extended-release Tablets, USP 750 mg 100 count in a 200 cc HDPE bottle heat sealed with 45 mm Polypropylene closure.

At end of 3 months, Trial 2 data (in Table 3) with addition of Oxygen Scavenger (Pharmakeep®) alone into the packaged product of Metformin Hydrochloride Extended-Release tablets reduces the formation of NDMA. Trial 2 Stability study was carried out providing two (2) Oxygen Scavenger (Pharmakeep® each unit weighing 2.15 g with 2 units weighing total of 4.30 g) to packaged product Metformin Hydrochloride Extended-release Tablets, USP 750 mg 100 count in a 200 cc HDPE container heat sealed with 45 mm Polypropylene closure.

At end of 3 months, Trial 3 data (in Table 3) with addition of desiccant (60SG/40CB) alone without oxygen scavenger into packaged product of Metformin Hydrochloride Extended-Release tablets significantly reduces the formation of NDMA. Trial 3 Stability study was carried out providing 2 g desiccant (2 units i.e. cans weighing 1 g each with total 2 g Desiccant having by weight 60% silica gel and 40% activated carbon) to packaged product Metformin Hydrochloride Extended-release Tablets, USP 750 mg 100 count in a 200 cc HDPE bottle heat sealed with 45 mm Polypropylene closure.

At end of 3 months, Trial 4 control data (in Table 3) demonstrates that presence of moisture and air/oxygen into the packaged product facilitates the formation of NDMA Impurity in Metformin Hydrochloride Extended-Release tablets. Trial 4 control stability study was carried out without any desiccant and without any oxygen scavenger on packaged product. At the end of 3 months of stability studies, the NDMA Impurity levels of packaged product with either the desiccant and/or oxygen scavenger were found to be controlled within the stability study specifications (NMT 30 μg/kg).

Based on further stability studies up to 6 months as shown in Table 4 below, values of NDMA for samples of Trial 1 with 2 desiccants and Trial 2 with 2 desiccants at bottom are lower compared to Trial 3 without oxygen scavenger & without desiccant. Trial 1 and 2 with desiccants after exposure of the samples at 40° C./75% RH for 3 months and 30° C./65% RH for 6 months still had lower NDMA values.

TABLE 4
N-nitrosodimethylamine (NDMA) Impurity Results at 0, 1, 2, 3 and 6 Months

| | | | |
|---|---|---|---|
| | | | Trial 3 |
| | Trial 1 | Trial 2 | Without Oxygen |
| | With 2 | With 2 Desiccants | Scavenger & |
| Time points | Desiccants | at bottom | without Desiccant |

Specification - NMT 30 μg/kg (LOQ 6.3 μg/kg)

| 0 Month | 10.5 μg/kg | | 12.1 μg/kg | | 48.0 μg/kg |
|---|---|---|---|---|---|
| 1 Month (25 ± 2° C. temperature/ | Below LOQ | | Below LOQ | | 45.9 μg/kg |
| 60 ± 5% relative humidity) | (3.4 μg/kg) | | (1.3 μg/kg) | | |
| 1 Month (30 ± 2° C. temperature/ | 14.8 | μg/kg | 13.4 | μg/kg | 61.1 μg/kg |
| 65 ± 5% relative humidity) | | | | | |
| 1 Month (40 ± 2° C. temperature/ | 10.9 | μg/kg | 11.8 | μg/kg | 62.5 μg/kg |
| 75 ± 5% relative humidity) | | | | | |
| 2 Months (25 ± 2° C. temperature/ | 8.4 | μg/kg | 8.2 | μg/kg | 48.7 μg/kg |
| 60 ± 5% relative humidity) | | | | | |
| 2 Months (30 ± 2° C. temperature/ | 15.6 | μg/kg | 10.3 | μg/kg | 61.6 μg/kg |
| 65 ± 5% relative humidity) | | | | | |
| 2 Months (40 ± 2° C. temperature/ | 10.1 | μg/kg | 8.9 | μg/kg | 87.7 μg/kg |
| 75 ± 5% relative humidity) | | | | | |
| 3 Months (25 ± 2° C. temperature/ | 6.7 | μg/kg | Below LOQ | | 13.6 μg/kg |
| 60 ± 5% relative humidity) | | | (4.7 μg/kg) | | |
| 3 Months (30 ± 2° C. temperature/ | 6.9 | μg/kg | Below LOQ | | 17.8 μg/kg |
| 65 ± 5% relative humidity) | | | (4.1 μg/kg) | | |
| 3 Months (40 ± 2° C. temperature/ | 8.3 | μg/kg | 8.6 | μg/kg | 48.1 μg/kg |
| 75 ± 5% relative humidity) | | | | | |
| 6 Months (25 ± 2° C. temperature/ | 8.8 | μg/kg | 13.5 | μg/kg | 49.4 μg/kg |
| 60 ± 5% relative humidity) | | | | | |
| 6 Months (30 ± 2° C. temperature/ | 10.5 | μg/kg | 7.4 | μg/kg | 73.0 μg/kg |
| 65 ± 5% relative humidity) | | | | | |

Table 4 N-nitrosodimethylamine (NDMA) values was measured by the following non-limiting technique as known in the art for measuring N-nitrosodimethylamine impurities in Metformin raw material and Drugs containing Metformin with the following conditions and equipments.

### Chromatographic Conditions:

Column: Thermo Accucore PFP, 2.6 μm, 100 mm×4.6 mm
Injection volume: 3 μL
Run Time: 15 Minutes
Flow Rate: 0.25 mL/minute
Autosampler Temp.: 21° C.
Column Temp: 35° C.
Mobile Phase: Mobile phase A—0.1% formic acid in water Mobile phase B—Acetonitile
Needle wash: Water:Methanol (80:20)
Rotor seal wash: 10% Methanol in water
   Mass spectrometer Conditions:

### Instrument

### Q Exactive Plus Mass Spectrometer (Thermo Fisher Scientific™)

HESI Source Settings (apply to both negative and positive modes)

Note: Set the Ion source probe at ‘C’ position. Ion source parameters can be adjusted to achieve the desired sensitivity.

ESI Source

| Ion source type | HESI | |
|---|---|---|
| Sheath Gas Flow Rate | 55 arbitrary units | |
| Aux Gas Flow Rate | 15 arbitrary units | |
| Sweep Gas Flow Rate | 0 | units |
| Spray Voltage | 3500 | V |
| Capillary Temp. | 250° | C. |
| S-Lens | 55 | |
| Aux Gas Heater Temp. | 425° | C. |

### CALCULATIONS

$$Nitrosamine Impurity ( ng / g ) = \frac{Asp l}{As} \times Cs \times \frac{V}{W} \times \frac{Purity}{100} \times 1000$$

Nitrosamine impurity refers to NDMA
Aspl=Area ratio of the nitrosamine impurity and NDMA D6 impurity peak in the sample solution.
As=Average area ratio (n=6) of the nitrosamine impurity and NDMA D6 peak from the first six consecutive injections of the standard solution.
Cs=Concentration of the nitrosamine impurity in the standard solution (3.0 ng/ml)
W=Weight of Sample weight (mg)
V=Volume of sample solution (mL)

While typical embodiments have been set forth for the purpose of illustration, the foregoing description should not be deemed to be a limitation on the scope of the invention. Accordingly, various modifications, adaptations, and alternatives may occur to one skilled in the art without departing from the spirit and scope of the present invention

1. A pharmaceutical package comprising:
a pharmaceutical solid dosage packaging container comprising
a desiccant disposed in the container;
a pharmaceutical product comprising an active pharmaceutical ingredient disposed in the container;
wherein the weight ratio of desiccant to active pharmaceutical ingredient is at least 1:150; and
the pharmaceutical product has an N-nitrosodimethylamine (NDMA) level of no more than 30 μg NMDA per kg of pharmaceutical product for at least 3 months under accelerated storage conditions at about 38° C.-42° C. and about 70-80% relative humidity or at least two years at room temperature.

2. The pharmaceutical package of claim 1, wherein the active pharmaceutical ingredient comprises at least one member selected from biguanides.

3. The pharmaceutical package of claim 1, wherein the active pharmaceutical ingredient comprises metformin.

4. The pharmaceutical package of claim 1, wherein the weight ratio of desiccant to active pharmaceutical ingredient is at least 1:75.

5. The pharmaceutical package of claim 1, wherein the desiccant comprises at least one member selected from silica gel, clay, molecular sieves, and combinations thereof.

6. The pharmaceutical package of claim 1, wherein the desiccant comprises silica gel.

7. The pharmaceutical package of claim 1, wherein the desiccant comprises activated charcoal.

8. The pharmaceutical package of claim 1, wherein the desiccant comprises a plurality of desiccants which differ from each other.

9. The pharmaceutical package of claim 1, wherein the active pharmaceutical ingredient comprises Metformin;
the desiccant comprises by weight about 55-65% Silica Gel (SiO₂) and about 35-45% activated Carbon; and
the weight ratio of desiccant to metformin is at least 1:75.

10. The pharmaceutical package of claim 1, wherein the active pharmaceutical ingredient comprises Metformin;
the desiccant substantially comprises by weight about 55-65% Silica Gel (SiO₂) and about 35-45% activated Carbon; and
the weight ratio of desiccant to Metformin is at least 2:75.

11. The pharmaceutical package of claim 1, further comprising an oxygen scavenger disposed in the container; and
a weight ratio of oxygen scavenger to active pharmaceutical ingredient is at least 1:150.

12. A pharmaceutical package comprising:
a pharmaceutical solid dosage packaging container comprising
a desiccant disposed in the container, wherein the desiccant comprises by weight about 55-65% Silica Gel (SiO₂) and about 35-45% activated Carbon;
pharmaceutical product comprising Metformin active pharmaceutical ingredient disposed in the container; and
a weight ratio of desiccant to Metformin active pharmaceutical ingredient of at least 1:150; and
wherein the pharmaceutical product has an N-nitrosodimethylamine (NDMA) level of no more than 30 μg NMDA per kg of pharmaceutical product for at least 3 months under accelerated storage conditions at about 38° C.-42° C. and about 70-80% relative humidity or at least two years at room temperature.

13. The pharmaceutical package of claim 12, wherein the desiccant comprises by weight about 55-65% Silica Gel (SiO₂) and about 35-45% activated Carbon.

14. The pharmaceutical package of claim 12, wherein the desiccant comprises a plurality of desiccants which differ from each other.

15. A method of reducing formation of N-nitrosodimethylamine (NDMA) in a pharmaceutical product comprising:
providing a desiccant to a container comprising
pharmaceutical product comprising an active pharmaceutical ingredient in a weight ratio of the desiccant to the active pharmaceutical ingredient of at least 1:150 to maintain an NDMA level of no more than 30 ug NMDA per kg of the pharmaceutical product for at least 3 months under accelerated storage conditions at about 38° C.-42° C. and about 70-80% relative humidity or at least two years at room temperature;
detecting the amount of NDMA in a container comprising the active pharmaceutical ingredient without a desiccant at time zero; and detecting the amount of NDMA in the container comprising the active pharmaceutical ingredient without desiccant after 30 days; and
comparing the amount of NDMA in the container comprising the active pharmaceutical ingredient and desiccant with the amount of NDMA in the container comprising the active pharmaceutical ingredient without desiccant.

16. The method of claim 15, wherein the active pharmaceutical ingredient comprises Metformin.

17. The method of claim 15, wherein the desiccant is provided in a weight ratio of desiccant to active pharmaceutical ingredient of at least 1:75.

18. The method of claim 15, wherein the desiccant comprises activated charcoal.

19. The method of claim 18, where the weight ratio of activated charcoal to the active pharmaceutical ingredient is greater than or equal to 1:225.

20. The method of claim 15, wherein the desiccant provided comprises silica gel.
